(19) United States
(12) Patent Application Publication
(10) Pub. No.: US 2010/0268303 A1
(43) Pub. Date: Oct. 21, 2010

(54) ESTABLISHING A COMMUNICATION SESSION BETWEEN AN IMPLANTABLE MEDICAL DEVICE AND AN EXTERNAL DEVICE USING A BURST PERIOD AND A SNIFF INTERVAL

(76) Inventors: William J. Mitchell, Eden Prairie, MN (US); Earle T. Roberts, Maple Grove, MN (US); David S. Slack, Plymouth, MN (US)

Correspondence Address: Medtronic - WK, P.O. Box 71355, Marietta, GA, 30007-1355, US

(21) Appl. No.: 12/426,261

(22) Filed: Apr. 19, 2009

(51) Int. Cl. A61N 1/08

(52) U.S. Cl. 607/60

(57) ABSTRACT

Communication sessions between an external device and an implantable medical device are established by the initiating device periodically transmitted bursts of energy and with the device that is the target of the bursts periodically sniffing for the transmitted bursts of energy. A silent period between bursts may be established and the sniff interval is chosen to avoid repetitively sniffing during the silent period. The length of the bursts may be chosen to reduce the delay in establishing the communications and/or to reduce the power consumption. The implantable medical device may use multiple modes of operation where in some modes the implantable medical device sniffs and in another mode the implantable medical device transmits bursts. The sniff interval for the implantable medical device may vary depending upon the mode. The burst length may vary depending upon whether the external device or the implantable device is transmitting the bursts.

### TECHNICAL FIELD

Embodiments relate to establishing a communication session between an external device and an implantable medical device. More particularly, embodiments relate to transmitting bursts for periods of time and sniffing for the bursts at intervals of time to establish the communication session.

### BACKGROUND

External devices and implantable medical devices (IMD) occasionally have a need to communicate. For instance, a clinician may need to review information stored within an IMD in order to assess a particular therapy and/or to modify therapy parameters. Likewise, a patient may need to increase or decrease the degree of therapy being received in some cases such as for pain alleviation. The IMD, such as a stimulator, drug delivery pump, and the like, may transfer requested information to the external device, such as a device programmer or a patient therapy manager (PTM), where it can be displayed, manipulated, and returned to the IMD.

The communication session between the external device and the IMD has typically used near field telemetry. A programming head, wand, or other tool is held in close proximity to the IMD while a signal is emitted from the wand. The IMD continuously sniffs for the signal and then responds to the external device via near field signaling to the wand upon detecting the signal. The near field signaling, particularly the sniffing for near field signals, requires a relatively small amount of power which has allowed the IMD to continuously sniff. Furthermore, the length of the bursting from the external device may not be constrained other than if there are power concerns such as for the PTM. However, even for the PTM, the IMD continuously sniffs and therefore quickly hears the burst once the PTM begins sending bursts.

Radio frequency (RF) communication links are now available between external devices and IMDs as a replacement for near field telemetry. The RF communication links provide some advantages over near field telemetry. For instance, RF communication links can be more convenient because the distance between the RF antenna of the external device and the integral RF antenna of the IMD can be separated at significantly greater distances than the distance of the wand to the IMD for near field telemetry.

While RF communication links are advantageous, there are drawbacks. For instance, government regulations typically apply to RF communications between external devices and IMDs. In the case of the Medical Implant Communications Service (MICS) band for instance, Federal Communications Commission (FCC) regulations call for a continuous burst length of no more than 4.8 seconds, with at least a 200 millisecond delay after each burst during which the bursting device must not transmit RF energy. During this silent period, the sniffing device will not receive a burst, and any sniffing during the silent periods wastes some amount of energy of the sniffing device, typically the IMD.

The IMD has a limited amount of available energy, and sniffing for RF energy requires a considerable amount of energy that prevents the IMD from continuously sniffing. Therefore, it is possible for the IMD to often attempt a sniff during silent periods, which increases the likelihood of wasting energy with unsuccessful sniffs. Additionally, sniffing during silent periods extends the amount of time and number of bursts necessary to establish the communication session. This may also be a concern particularly where the bursting device also has limited power, as with a PTM or in situations where the IMD is the bursting device.

### SUMMARY

Embodiments address issues such as these and others by providing for an interval between sniffs that reduces the likelihood of a relatively high number of unsuccessful sniffs. The embodiments provide for different sniff intervals depending upon the mode of the implantable medical device at the time of the sniffing and depending upon whether the implantable device or the external device is sniffing.

Embodiments provide a method of establishing communication between an external device and an implantable medical device. In relation to this method, the external device sends a burst for a first length of time with an interval between bursts of a second length of time that is shorter than the first length of time until receiving a response from the implantable medical device. The method involves sniffing by the implantable medical device for a third length of time with an interval between sniffs of a fourth length of time until receiving a burst from the external device during the sniffing. The third length of time is less than the second length of time, and the fourth length of time is less than the first length of time and greater than the second length of time. After receiving the burst, the implantable medical device provides the response.

Embodiments provide another method of establishing communication between an external device and an implantable medical device. In relation to this method, the external device sends a burst for a first length of time with an interval between bursts of a second length of time that is shorter than the first length of time until receiving a response from the implantable medical device. The method involves sniffing by the implantable medical device for a third length of time with an interval between sniffs of a fourth length of time until receiving a burst from the external device during the sniffing. The third length of time is less than the second length of time, and the fourth length of time is greater than the sum of the first length of time and the second length of time and is not a multiple of the sum of the first length of time and the second length of time. After receiving the burst, the implantable medical device provides the response from the implantable medical device.

Embodiments provide another method of establishing communication between an external device and an implantable medical device. In relation to this method, the external device sniffs for a first length of time with an interval between sniffs of a second length of time that is longer than the first length of time until receiving a burst from the implantable medical device. The method involves sending a burst by the implantable medical device for a third length of time with an interval between bursts of a fourth length of time until receiving a response from the external device. The third length of time is less than the second length of time and greater than the first length of time, and the fourth length of time is greater than the second length of time. The implantable device receives the response from the external device.

Embodiments provide an implantable medical device that includes communication circuitry that exchanges communication signals with an external device. The implantable medical device also includes a processor that implements at least a first and a second mode to control the communication circuitry. In relation to this implantable medical device and during the first and second modes, the external device sends a burst for a first length of time with an interval between bursts of a second length of time that is shorter than the first length of time until receiving a response from the implantable medical device.

For this embodiment, during the first mode the processor triggers sniffing by the communication circuitry for a third length of time with an interval between sniffs of a fourth length of time until receiving a burst from the external device during the sniffing. The third length of time is less than the second length of time, and the fourth length of time is less than the first length of time and greater than the second length of time. During the second mode the processor triggers sniffing by the communication circuitry for the third length of time with an interval between sniffs of a fifth length of time until receiving the burst from the external device during the sniffing. The fifth length of time is greater than the sum of the first length of time and the second length of time and is not a multiple of the sum of the first length of time and the second length of time. After receiving the burst, the processor provides the response from the communication circuitry.

Embodiments provide an external device for communication with an implantable medical device. The external device includes communication circuitry that exchanges communication signals with the implantable medical device. The external device also includes a processor that controls the communication circuitry. The processor triggers the communication circuitry to sniff for a first length of time with an interval between sniffs of a second length of time that is longer than the first length of time until receiving a burst from the implantable medical device. In relation to this external device, the implantable medical device sends a burst for a third length of time with an interval between bursts of a fourth length of time until receiving a response from the external device. The first length of time is less than the third length of time, and the second length of time is greater than the third length of time, less than the fourth length of time, and is not a multiple of a sum of the third length of time and the fourth length of time.

### DESCRIPTION OF THE DRAWINGS

FIG. 1 shows an operating environment for illustrative embodiments that establish communication sessions by using a burst period and sniff interval.

FIG. 2 shows an illustrative external device embodiment that communicates with an implantable medical device (IMD).

FIG. 3 shows an illustrative IMD embodiment that communicates with an external device.

FIG. 4 shows an example of operational flow of an external device embodiment that sends bursts and delays between bursts.

FIG. 5 shows an example of operational flow of a device embodiment that sniffs at intervals.

FIG. 6 shows a timeline where an IMD embodiment sniffs at an interval for a normal state.

FIG. 7 shows a timeline for three scenarios where an IMD embodiment sniffs at an interval for a reduced power state.

FIG. 8 shows an example of operational flow of an IMD embodiment that sends bursts and delays between bursts.

FIG. 9 shows a timeline where an IMD embodiment sends bursts and delays between bursts during an event mode while an external device embodiment sniffs at an interval.

FIG. 10 shows a chart of three scenarios for an external device embodiment receiving a burst from an IMD embodiment in the event mode.

### DETAILED DESCRIPTION

Communication sessions between embodiments of external devices and embodiments of implantable medical devices (IMD) are established by bursts of electromagnetic energy sent from an initiating device and by sniffs for such energy by a listening device. In some instances, the initiating device is the external instrument and the listening device is the IMD, but other instances call for the IMD to transmit bursts while the external device sniffs for the transmission. The bursts of electromagnetic energy are transmitted by the initiating device with a silent period between bursts while the sniffs of the listening device occur at intervals that attempt to reduce the number of sniffs that occur during silent periods.

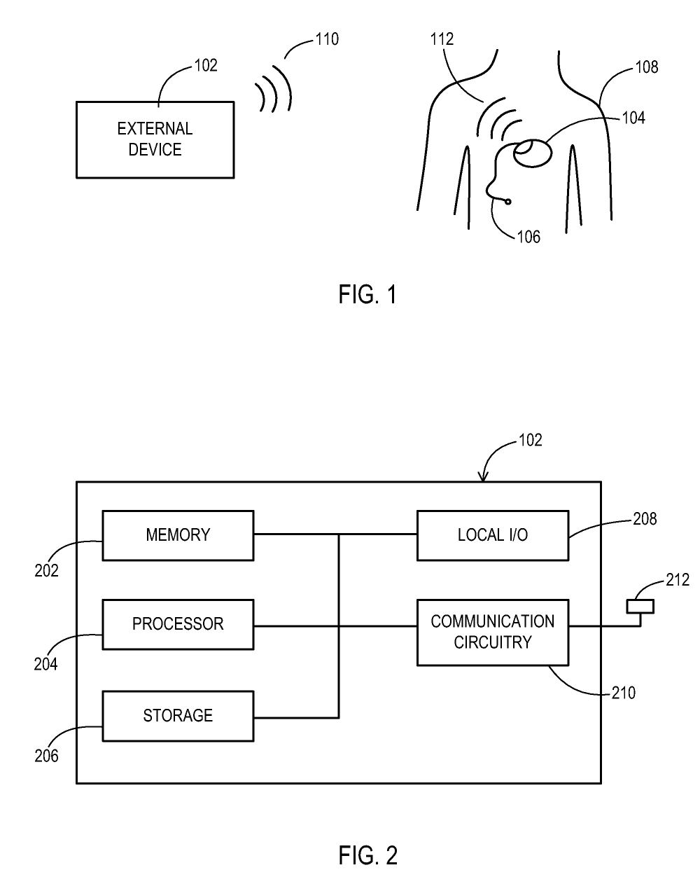

FIG. 1 shows an external device 102 in communication with an IMD 104 that is implanted within a patient 108. The external device 102 may be one of various device types, such as a device programmer or a patient therapy manager (PTM). Likewise, the IMD 104 may be of various device types as well, such as a stimulator, a monitor, a drug delivery pump, a data collector, and so forth. The IMD 104 may have medical components 106, such as stimulation leads, monitoring leads, catheters, and the like.

The external device 102 and the IMD 104 typically communicate through a form of telemetry. In the case of a wireless communication link, wireless signals 110 are sent by the external device 102 and are received by the IMD 104. Likewise, wireless signals 112 are sent by the IMD 104 and are received by the external device 102. As an example, the telemetry may use radio frequency (RF) signaling where an antenna of the external device 102 and the IMD 104 are separated by a larger distance than occurs with near field telemetry to provide added convenience.

Typically, the external device 102 initiates a communication session with the IMD 104 by attempting to wake up the telemetry of the IMD 104. The external device 102 sends bursts of energy for a first length of time and then delays for a second length of time before sending the next burst. Likewise, the IMD 104 sniffs for a third length of time and delays for a fourth length of time before sniffing again. This cycle repeats for both devices until the IMD 104 sniffs the burst of energy from the external device 102 and sends a response signal. The selection of those four lengths of time dictates the number of sniffs that will be needed to receive a burst for the many scenarios that may occur.

According to one or more of the embodiments, the IMD 104 may also operate in a mode that causes the IMD 104 to attempt to wake-up the external device 102. For instance, the IMD 104 may have a medical emergency mode whereby it detects a medical emergency and attempts to notify the external device 102 of the emergency. In that case, the IMD 104 sends bursts of energy for a first length of time and then delays for a second length of time before sending the next burst. The IMD 104 sends a burst for a third length of time and delays for a fourth length of time before bursting again. This cycle repeats for both devices until the external device 102 sniffs the burst of energy from the IMD 104 and sends a response signal. The selection of those four lengths of time also dictates the number of sniffs that will be needed to receive a burst for the many scenarios that may occur.

FIG. 2 shows components of one example of the external device 102. The external device 102 includes a memory 202, a processor 204, and a storage device 206. The external device 102 may also include local input/output (I/O) ports 208 such as to provide local screen displays and to receive user input via keyboard, mouse, and so forth. The external device 102 also includes communication circuitry 210 used to establish the telemetry to the IMD 104. The communication circuitry 210 may drive a signal propagation tool 212, such as an RF antenna.

The memory 202 may be used to store information in use by the processor 204. For instance, the memory 202 may store therapy parameters that are input by a clinician or patient that are to be loaded into the IMD 104. The memory 202 may also store programming that is used by the processor 204 to control the bursting and sniffing actions of the external device 102. The memory 202 may be of various types, such as volatile, non-volatile, or a combination of the two.

The storage device 206 may be used to store information for a long term and may be of various types such as non-volatile so that the information is retained when the external device 102 is powered off. The storage device 206 may also store programming for the processor 204 that is implemented to control the bursting and sniffing actions. Examples of the storage device 206 include electronic, magnetic, and optical drives. The storage device 206 and the memory 202 are both examples of computer readable media that may store information in the form of computer programming, data structures, and the like.

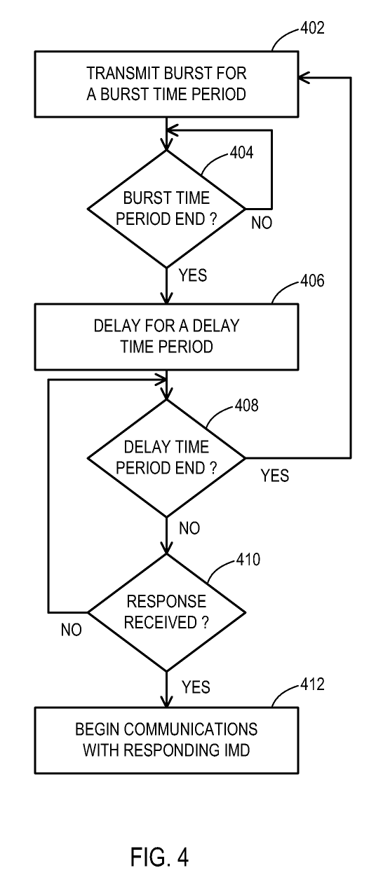

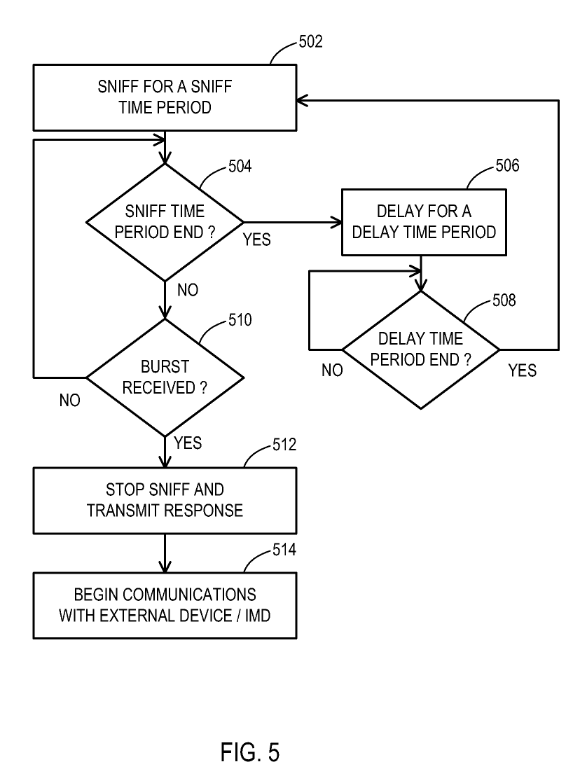

The processor 204 performs logical operations such as those of FIGS. 4 and 5 to allow communication sessions with the IMD 104 to be initiated. The processor 204 may be of various forms. For instance, the processor 204 may be a general-purpose programmable processor that executes software that is stored on the storage device 208 or elsewhere. Other examples include a dedicated purpose hardware circuit or hard-wired digital logic. The processor 204 may communicate with the various other components through one or more data buses.

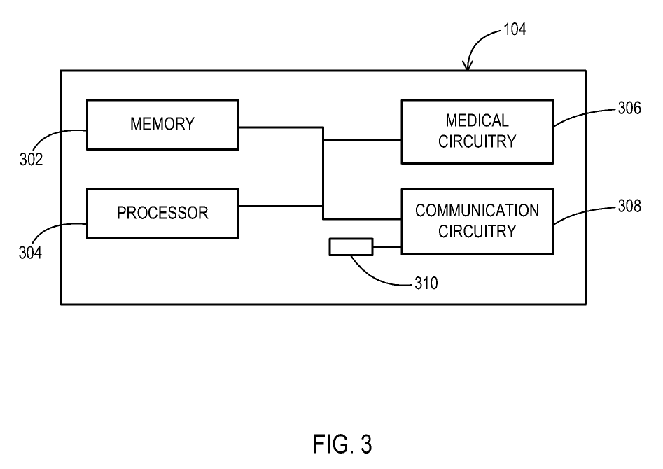

FIG. 3 shows components of one example of the IMD 104. The IMD 104 includes a memory 302 and a processor 304. The IMD 104 also includes medical circuitry 306 that performs a medical task such as stimulation, drug delivery, monitoring, and the like. The IMD 104 also includes communication circuitry 308 used to establish the telemetry to the external device 102. The communication circuitry 308 may drive a signal propagation tool 310, such as an integral RF antenna.

The memory 302 may be used to store information in use by the processor 306 such as programming and data values. The memory 302 may store additional information including therapy parameters that are used to control the medical circuitry 308. The memory 302 may be of various types such as volatile, non-volatile, or a combination of the two. The memory 302 is also an example of computer readable media that may store information in the form of computer programming, data structures, and the like.

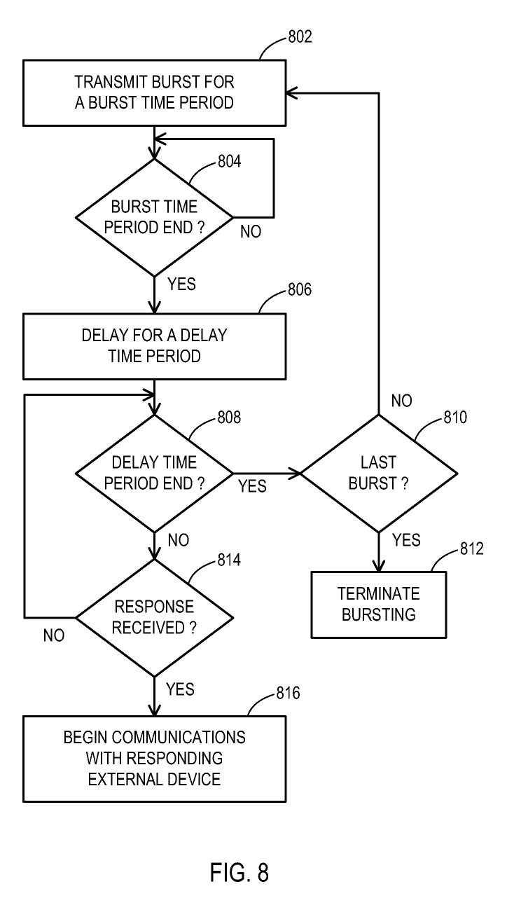

The processor 304 performs logical operations such as those of FIGS. 5 and 8 to allow communication sessions with the external device 102 to be initiated. The processor 304 may be of various forms like those discussed above for the processor 204 of the external device 102. The processor 304 may communicate with the various other components through one or more data buses.

FIG. 4 shows an example of logical operations that may be performed by the external device 102 when attempting to initiate a communication session with the IMD 104. The external device 102 begins transmitting a burst of energy for a first length of time at a transmit operation 402. The length of time may be relatively lengthy compared to the length of a sniff, and per the Federal Communications Commission (FCC) regulation for the Medical Implant Communications Service (MICS) band, may be a maximum of 4.8 seconds.

At a query operation 404, the external device 102 detects whether the period for bursting has ended. If not, then the bursting continues. If the period has ended, then the external device 102 begins to delay further bursting for a second length of time at a delay operation 406. This period of silence may be of various lengths but is typically much shorter than the burst length for an external device. Per the FCC regulation for the MICS band, this second length of time during which the external device 102 is silent should be at least 200 milliseconds for every burst/silence cycle.

At a query operation 408, the external device 102 detects whether the period for delaying the next burst has ended. If the period of delay has ended, then the external device 102 begins the next burst at the transmit operation 402. If the period of delay has not ended, then the external device 102 detects whether a response has been received from the IMD 104 at a query operation 410 that would indicate that the IMD 104 has sniffed during the preceding burst. If no response has been received, then the external device 102 continues to check for the end of the delay period at the query operation 408 and for an incoming response during the delay at the query operation 410.

Once a response is received during one of the delay periods, then the external device 102 begins the communication session with the responding IMD at a communication operation 412. For instance, the external device 102 may send an interrogation signal to the implantable medical device 104 to obtain information or send therapy parameters to modify the therapy being provided.

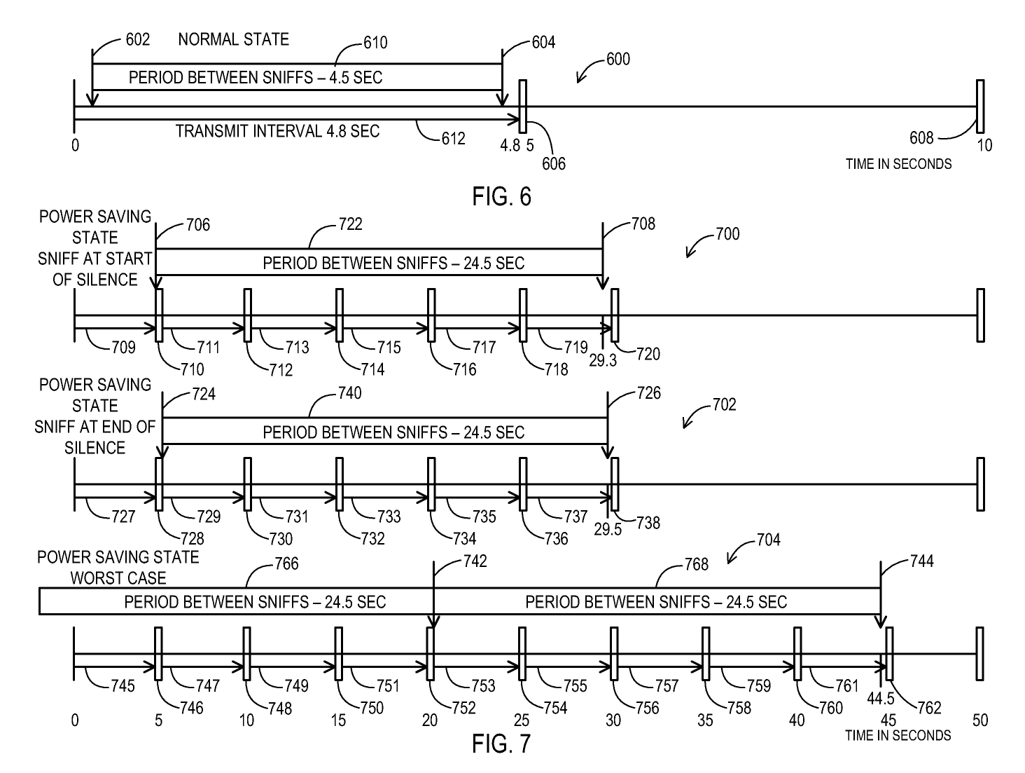

FIG. 5 shows an example of logical operations that may be performed by embodiments of the IMD 104 as well as one or more embodiments of the external device 102 when sniffing for bursts in order to wake-up at the appropriate time. The external device 102 may sniff for bursts during periods when the external device 102 is not sending bursts so that the external device 102 can be responsive to the IMD 104 when the IMD 104 enters a mode that causes the IMD 104 to send bursts to initiate communications. While the same or similar logical operations may be employed by both the external device 102 and the IMD 104 when sniffing for bursts, the interval between sniffs may be entirely different, and the length of the bursts being sniffed may also be entirely different. Furthermore, the sniff interval may change depending upon a mode of operation that the device is currently using. Examples of various modes of operation are discussed below with reference to FIGS. 6, 7, and 9.

At a sniff operation 502, the external device 102 or the IMD 104 sniffs for a sniff time period. The sniff time period may be a relatively small amount of time, such as 50 milliseconds, since a burst may be detected over a span of even less than 50 milliseconds. It is determined whether the end of the sniff time period has occurred at a query operation 504. If the end of the sniff period has been reached, then the external device 102 or the IMD 104 delays for a delay time period at a delay operation 506. Here, the external device 102 or the IMD 104 turns off the receiver to conserve energy.

During the delay time period, the external device 102 or the IMD 104 monitors for the end of the delay time period at a query operation 508. Once the end of the delay time period is reached, then the external device 102 or the IMD 104 begins to sniff again for the sniff time period at the sniff operation 502. This delay time period defines the sniff interval.

The sniff interval for the external device 102 or the IMD 104 may be set in an attempt to avoid repeatedly sniffing during the silent period between bursts. For a bursting device that transmits the full 4.8 seconds allowed in the MICS band, which is likely to be the external device 102, then the sniffing device such as the IMD 104 may employ a sniff interval of less than 4.8 seconds in some instances such as where a more immediate wake-up is desired and/or may employee a sniff interval of greater than 4.8 seconds in other instances where the wake-up period can be lengthier.

Returning to the query operation 504, when the sniff interval has not yet ended, then the external device 102 or the IMD 104 detects whether a burst is received by the sniff at a query operation 510. If not, the external device 102 or the IMD 104 continues to sniff. When a query operation 510 finds that a burst has been received, then in this example, the external device 102 or the IMD 104 stops checking for whether a burst has been received and instead sniffs until recognizing the end of the burst. Then the external device 102 or the IMD 104 stops sniffing and transmits a response to the bursting device at a transmit operation 512. The response acknowledges that the bursting device has been heard and that the responding device is ready to begin communicating data. The external device 102 or the IMD 104 then begins communications with the bursting device at a communications operation 514.

FIG. 6 shows a timeline 600 that pertains to the external device 102 in a bursting mode where bursts are being periodically transmitted in an attempt to wake-up the IMD 104 and establish communications. In this example, the external device 102 is transmitting the maximum burst length of 4.8 seconds that is allowed for the MICS band which is indicated by a horizontal arrow 612. The silent periods between bursts 612 are shown as the bars 608, 610.

The IMD 104 is sniffing at a designated sniff interval for a current mode of operation. In this example, the IMD 104 is in a normal operating mode such as where the IMD 104 is installed for a patient and no medical emergency is taking place. In this embodiment of the IMD 104, the sniff interval for the normal mode is set to a value that is less than the burst length of the external device 102. For this example, the sniff interval is set to 4.5 seconds for a burst length of 4.8 seconds. The sniffs by the IMD 104 are shown as vertical arrows 602, 604, with the delay period between sniffs as the horizontal bar 610.

As shown, the first sniff 602 falls during the burst 612. Therefore, it is likely that the IMD 104 receives the burst and can respond. However, if that first sniff fails, a second sniff 604 occurs also during the burst 612 to increase the likelihood that the IMD 104 receives the burst. With the sniff interval being less than the burst length, if the first sniff had fallen during a silent period, such as during the silent period 606, then the next sniff would have occurred prior to the next silent period 608 such that the burst would have been received. Therefore, it is likely that the IMD 104 may receive the burst by the second sniff even in a worst case scenario where the first sniff occurs during any part of the silent period 606.

FIG. 7 shows a comparison of three time line scenarios during which the IMD 104 is in a power-saving mode of operation. A power-saving mode of operation may occur for various reasons. For instance, the IMD 104 may be placed into a power-saving mode at the time of manufacture to preserve battery life while the IMD 104 is stored and transported prior to implantation. As another instance, the IMD 104 may detect a low energy condition at some point in time after being implanted and may enter the power-saving mode to conserve power and maintain therapy for a longer period of time.

In the power-saving mode, the IMD 104 may sniff at an interval that is longer than the sniff interval that is specified for the normal mode of operation as shown in FIG. 6. During the power-saving mode, it may continue to be desirable to avoid repeatedly sniffing during silent periods between bursts. Therefore, the sniff interval may be set to a length of time that is greater than the burst length but is not a multiple of the burst length plus the silent period to avoid always sniffing during the silent period. In this manner, it is likely that the IMD 104 will receive the burst on the first or second sniff.

In the first scenario 700, the IMD 104 performs a first sniff 706. Had this sniff occurred during a first burst 709 or a second burst 711, the IMD 104 could have heard the burst 709 or 711 and responded. However, this first scenario 700 demonstrates the first sniff 706 occurring at the beginning of the first silent period 710 between bursts which is at 4.8 seconds from zero. The IMD 104 has a delay period 722 in this example of 24.5 seconds, which is significantly longer than the burst length of 4.8 seconds in an attempt to save more power of the IMD 104. However, this sniff interval of 24.5 seconds is not a multiple of the burst period which is the burst length plus the silent period. In this example, the burst period is 4.8 seconds plus 200 milliseconds, or 5.0 seconds.

As a result of the sniff interval not being a multiple of 5 seconds, the next sniff occurs at 29.3 seconds from zero, which falls within a burst 719 and just before a silent period 720. While bursts 713, 715, and 717 and corresponding silent periods 712, 714, 716, and 718 are skipped by the lengthier sniff interval, the second sniff 708 receives the burst 719 so that the IMD 104 can respond and communications can begin.

In the second scenario 702, the IMD 104 performs a first sniff 724. Had this sniff occurred during a first burst 727 or a second burst 729, the IMD 104 could have heard the burst 727 or 729 and responded. However, this second scenario 702 demonstrates the first sniff 724 occurring at the end of the first silent period 728 between bursts which is at 5.0 seconds from zero. The IMD 104 has a delay period 740 in this example that is also 24.5 seconds and therefore is not a multiple of the burst period of 5.0 seconds.

As a result of the sniff interval not being a multiple of 5 seconds, the next sniff occurs at 29.5 seconds from zero, which falls within a burst 737 and just before a silent period 738. While bursts 729, 731, 733, and 735 and corresponding silent periods 730, 732, 734, and 736 are skipped by the lengthier sniff interval, the second sniff 726 receives the burst 737 so that the IMD 104 can respond and communications can begin.

In the third scenario 704, the IMD 104 performs a first sniff 742 after four bursts 745, 747, 749, and 751 and corresponding silent periods 746, 748, and 750 have ended. Had this sniff occurred during the fourth burst 751 or a firth burst 753, the IMD 104 could have heard the burst 751 or 753 and responded. However, this third scenario 704 demonstrates the first sniff 742 occurring at the end of the fourth silent period 752 at 20.0 seconds from zero and may be considered a worst case. The IMD 104 has a delay period 766, 768 in this example that is also 24.5 seconds and therefore is not a multiple of the burst period of 5.0 seconds.

As a result of the sniff interval not being a multiple of 5 seconds, the next sniff occurs at 44.5 seconds from zero, which falls within a burst 761 and just before a silent period 762. While intervening bursts 753, 755, 757, and 759 and corresponding silent periods 754, 756, 758, and 760 are skipped by the lengthier sniff interval, the second sniff 744 receives the burst 761 so that the IMD 104 can respond and communications can begin.

FIG. 8 shows an example of logical operations that may be performed by the IMD 104 during a medical event mode which necessitates an attempt to initiate a communication session with the external device 102. For instance, the IMD 104 may detect an emergency that qualifies for the medical event mode to be used and may switch to that mode automatically. The IMD 104 begins transmitting a burst of energy for a first length of time at a transmit operation 802.

As with the burst length by the external device 102 in FIG. 4, the burst length by the IMD 104 may be substantial, and per the FCC regulation for the MICS band, may be a maximum of 4.8 seconds with at least 200 milliseconds of silence for every 5 second burst/silence cycle. However, it may be desirable to use a burst length of less than 4.8 seconds and a silent period of greater than 200 milliseconds to conserve the available energy of the IMD 104. Furthermore, because the IMD 104 may enter the medical event mode automatically and with no knowledge of whether an external device 102 is present to receive the bursts, there may in fact be no external device 102 nearby. Therefore, it may be desirable to limit the number of bursts so that once that limit is reached the IMD 104 automatically exits the medical event mode and stops sending bursts. By exiting the medical event mode, energy is not wasted by continuing to send bursts when no response will be provided.

At a query operation 804, the IMD 104 detects whether the period for bursting has ended. If not, then the bursting continues. If the period has ended, then the IMD 104 begins to delay further bursting for a second length of time at a delay operation 806. This period of silence may be of various lengths but may be longer than the burst length to further assist in conserving energy.

At a query operation 808, the IMD 104 detects whether the period for delaying the next burst has ended. If the period of delay has ended, then the IMD 104 detects whether the last permissible burst for the medical event mode has been sent at a query operation 810. If not, then the IMD 104 begins the next burst at the transmit operation 802. If the last permissible burst has been sent, then medical event mode is ended at a termination operation 812 where the IMD 104 then returns to the normal mode where sniffing in accordance with the operations of FIG. 5 begins.

Returning to the query operation 808, if the period of delay has not ended, then the IMD 104 detects whether a response has been received from the external device 102 at a query operation 814 that would indicate that the external device 102 has sniffed during the preceding burst. If no response has been received, then the IMD 104 continues to check for the end of the delay period at the query operation 808 and for an incoming response during the delay at the query operation 814.

Once a response is received during one of the delay periods, then the IMD 104 begins the communication session with the responding external device 102 at a communication operation 816. For instance, the IMD 104 may await an interrogation signal from the external device 102 to provide information about the medical event to the external device 102.

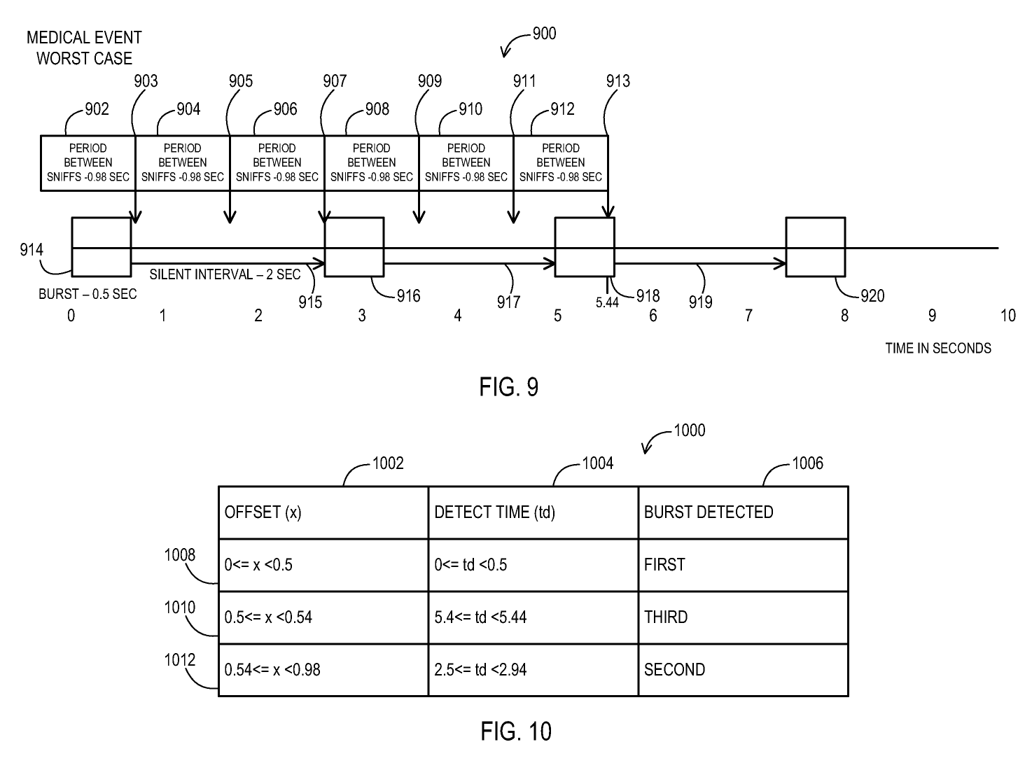

FIG. 9 shows a timeline scenario 900 for an attempt by the IMD 104 in the medical event mode to initiate communications with the external device 102. In this example, the IMD 104 is configured to attempt bursts over a span of 10 seconds. Furthermore, burst length is configured to be less than the silent period and also less than the sniff interval being used by the external device 102. In this particular example, the burst length of the IMD 104 is set at 0.5 seconds, the silent interval of the IMD 104 between bursts is set at 2.0 seconds, the sniff length of the external device 102 is about 50 milliseconds, and the sniff interval of the external device 102 is 0.98 seconds. Because the sniff interval is not a multiple of the burst length plus the silent interval, the sniffs should not repetitively fall during the silent interval.

What may be considered a worst case scenario is shown in FIG. 9 where the first sniff 903 occurring after the first burst 914 has started occurs immediately after the first burst 914 ends. Had the sniff 903 occurred sooner, during the burst 914, then the external device 102 could have responded with no further bursts and sniffs being needed. However, the sniff 903 occurs during the first silent interval 915 as the first burst 914 occurs entirely during the first sniff interval 902. The second sniff 905, second sniff interval 904, third sniff 907, and third sniff interval 906 also fall within this first silent interval 915.

The second burst 916 occurs just after the third sniff 907. However, in this worst case scenario, the second burst 916 occurs entirely during the fourth sniff interval 908. The fourth sniff 909 and fifth sniff 911 as well as the fifth sniff interval 910 occur during the second silent period 917, and the sixth sniff interval 912 begins during the second silent period 917. However, during the sixth sniff interval 912, the third burst 918 begins, and the sixth sniff 913 occurs during the third burst 918. Thus, while five sniffs by the external device 102 were unsuccessful, only three bursts by the IMD 104 were needed to establish communications. In this example, a fourth burst 920 may also be transmitted by the IMD 104 as a last effort to contact the external device 102 before exiting the medical event mode if no response has been received by the end of a third silent interval 919.

FIG. 10 shows a chart 1000 that pertains to the medical event mode where the IMD 104 is sending bursts and the external device 102 is presumably sniffing. This chart 1000 relates the offset between the start of the first burst by the IMD 104 and the first sniff by the external device 102 to the time from zero that the external device 102 sniffs during a burst. Zero is the time that the first burst begins. The three offset ranges of interest are shown in a first column 1002, while the three ranges of time when a sniff will occur during a burst are shown in a second column 1004. A third column 1006 indicates which burst is being detected.

A first row 1008 specifies a first offset range of 0 to less than 0.5 seconds. Here, the first sniff is occurring during the first burst. Thus, the detection time is also 0 to less than 0.5 seconds. A second row 1010 specifies a second offset range of 0.5 seconds to less than 0.54 seconds. Here, as in FIG. 9, the first sniff occurs just after the first burst has ended which extends the detection time to that range of 5.4 seconds to less than 5.44 seconds, and thus the third burst is detected. A third row 1012 specifies a third offset range of 0.54 seconds to less than 0.98 seconds, where it is noted that if the offset is 0.98 seconds or greater then the preceding sniff is occurring during the first burst because the sniff interval is 0.98 seconds. The detection time ranges from 2.5 seconds to less than 2.94 seconds, and thus the second burst is sniffed.

While embodiments have been particularly shown and described, it will be understood by those skilled in the art that various other changes in the form and details may be made therein without departing from the spirit and scope of the invention.

What is claimed is:

1. A method of establishing communication between an external device and an implantable medical device, wherein the external device sends a burst for a first length of time with an interval between bursts of a second length of time that is shorter than the first length of time until receiving a response from the implantable medical device, the method comprising:
sniffing by the implantable medical device for a third length of time with an interval between sniffs of a fourth length of time until receiving a burst from the external device during the sniffing, wherein the third length of time is less than the second length of time and wherein the fourth length of time is less than the first length of time and greater than the second length of time; and
after receiving the burst, providing the response from the implantable medical device.

2. The method of claim 1, further comprising:
upon receiving the burst during the sniffing, continuing to sniff to detect the end of the received burst; and
providing the response from the implantable device upon detecting the end of the received burst.

3. The method of claim 1, wherein the first length of time is 4.8 seconds and the second length of time is 0.2 seconds.

4. The method of claim 1, wherein the third length of time is 50 milliseconds.

5. The method of claim 1, wherein the fourth length of time is 4.5 seconds.

6. A method of establishing communication between an external device and an implantable medical device, wherein the external device sends a burst for a first length of time with an interval between bursts of a second length of time that is shorter than the first length of time until receiving a response from the implantable medical device, the method comprising:
sniffing by the implantable medical device for a third length of time with an interval between sniffs of a fourth length of time until receiving a burst from the external device during the sniffing, wherein the third length of time is less than the second length of time and wherein the fourth length of time is greater than the sum of the first length of time and the second length of time and is not a multiple of the sum of the first length of time and the second length of time; and
after receiving the burst, providing the response from the implantable medical device.

7. The method of claim 6, further comprising:
upon receiving the burst during the sniffing, continuing to sniff to detect the end of the received burst; and
providing the response from the implantable device upon detecting the end of the received burst.

8. The method of claim 6, wherein the first length of time is 4.8 seconds and the second length of time is 0.2 seconds.

9. The method of claim 1, wherein the third length of time is 50 milliseconds.

10. The method of claim 1, wherein the fourth length of time is 24.5 seconds.

11. A method of establishing communication between an external device and an implantable medical device, wherein the external device sniffs for a first length of time with an interval between sniffs of a second length of time that is longer than the first length of time until receiving a burst from the implantable medical device, the method comprising:
sending a burst by the implantable medical device for a third length of time with an interval between bursts of a fourth length of time until receiving a response from the external device, wherein the third length of time is less than the second length of time and greater than the first length of time and wherein the fourth length of time is greater than the second length of time; and
receiving the response at the implantable medical device from the external device.

12. The method of claim 11, wherein receiving the response occurs during the interval between bursts.

13. The method of claim 11, wherein the first length of time is 50 milliseconds and the second length of time is 0.98 seconds.

14. The method of claim 11, wherein the third length of time is 0.5 seconds.

15. The method of claim 11, wherein the fourth length of time is 2 seconds.

16. An implantable medical device, comprising:
communication circuitry that exchanges communication signals with an external device; and
a processor that implements at least a first and a second mode to control the communication circuitry, wherein during the first and second modes the external device sends a burst for a first length of time with an interval between bursts of a second length of time that is shorter than the first length of time until receiving a response from the implantable medical device, and
during the first mode, the processor triggers sniffing by the communication circuitry for a third length of time with an interval between sniffs of a fourth length of time until receiving a burst from the external device during the sniffing, wherein the third length of time is less than the second length of time and wherein the fourth length of time is less than the first length of time and greater than the second length of time,
during the second mode the processor triggers sniffing by the communication circuitry for the third length of time with an interval between sniffs of a fifth length of time until receiving the burst from the external device during the sniffing, wherein the fifth length of time is greater than the sum of the first length of time and the second length of time and is not a multiple of the sum of the first length of time and the second length of time, and
after receiving the burst, providing the response from the communication circuitry.

17. The implantable medical device of claim 16, wherein the processor further implements a third mode to control the communication circuitry, wherein during the third mode the external device sniffs for a sixth length of time with an interval between sniffs of a seventh length of time that is longer than the sixth length of time until receiving a burst from the implantable medical device,
during the third mode the processor triggers sending a burst by the communication circuitry for an eighth length of time with an interval between bursts of a ninth length of time until receiving a response from the external device, wherein the eighth length of time is less than the seventh length of time and greater than the sixth length of time and wherein the ninth length of time is greater than the seventh length of time, and
the processor receives the response from the external device through the communication circuitry.

18. The implantable medical device of claim 16, wherein upon the processor recognizing that the communication circuitry has received the burst during the sniffing, the processor triggers the communication circuitry to continue to sniff to detect the end of the received burst, and the processor provides the response to the external device through the communication circuitry upon detecting the end of the burst.

19. The implantable medical device of claim 16, wherein the first length of time is 4.8 seconds and the second length of time is 0.2 seconds.

20. The implantable medical device of claim 16, wherein the third length of time is 50 milliseconds.

21. The implantable medical device of claim 16, wherein the fourth length of time is 4.5 seconds.

22. The implantable medical device of claim 16, wherein the fifth length of time is 24.5 seconds.

23. The implantable medical device of claim 17, wherein the sixth length of time is 50 milliseconds, the seventh length of time is 0.98 seconds, the eight length of time is 0.5 seconds, and the ninth length of time is 2 seconds.

24. An external device for communication with an implantable medical device, comprising:
communication circuitry that exchanges communication signals with the implantable medical device; and
a processor that controls the communication circuitry, wherein the processor triggers the communication circuitry to sniff for a first length of time with an interval between sniffs of a second length of time that is longer than the first length of time until receiving a burst from the implantable medical device, while the implantable medical device sends a burst for a third length of time with an interval between bursts of a fourth length of time until receiving a response from the external device, wherein the first length of time is less than the third length of time, and wherein the second length of time is greater than the third length of time, is less than the fourth length of time, and is not a multiple of a sum of the third length of time the fourth length of time.

25. The external device of claim 24, wherein upon the processor recognizing that the communication circuitry has received the burst during the sniffing, the processor triggers the communication circuitry to continue to sniff to detect the end of the received burst, and the processor provides the response to the implantable medical device through the communication circuitry upon detecting the end of the burst.

26. The external device of claim 17, wherein the first length of time is 50 milliseconds, the second length of time is 0.98 seconds, the third length of time is 0.5 seconds, and the fourth length of time is 2 seconds.
